(19) United States
(12) Patent Application Publication
(10) Pub. No.: US 2024/0199652 A1
(43) Pub. Date: Jun. 20, 2024

(54) Preparation of Copper (I) 5-Nitrotetrazolate (DBX-1) from Bis(Ethylenediamine) Copper (II) Nitrotetrazolate

(71) Applicant: QINETIQ LIMITED, Farnborough Hampshire (GB)

(72) Inventors: Declan Haydn Williams, Sevenoaks Kent (GB); Javid Hamid, Sevenoaks Kent (GB); Robert Peter Claridge, Sevenoaks Kent (GB)

(21) Appl. No.: 17/905,546

(22) PCT Filed: Mar. 8, 2021
(86) PCT No.: PCT/EP2021/055708

§ 371 Date: Sep. 2, 2022

(30) Foreign Application Priority Data
Mar. 12, 2020 (GB) 2003580.4

(51) Int. Cl. C07F 1/08; C06B 41/00

(52) U.S. Cl. CPC C07F 1/08; C06B 41/00

(57) ABSTRACT

A method of making copper (I) 5-nitrotetrazolate (DBX-1) in isolated form comprising: a) reacting bis(ethylenediamine) copper (II) nitrotetrazolate with nitric acid or sulfuric acid to form a reaction product, subsequently b) reducing the reaction product of a) with ascorbate or ascorbic acid and reacting with copper chloride or copper sulfate to form copper (I) 5-nitrotetrazolate, and subsequently isolating the copper (I) 5-nitrotetrazolate (DBX-1) as a precipitate. The copper (I) 5-nitrotetrazolate produced by said method is useful as a primary explosive.

### CROSS-REFERENCE

This application is a 371 U.S. national phase of PCT/EP2021/055708, filed Mar. 8, 2021, which claims priority from GB patent application no. 2003580.4, filed Mar. 12, 2020, both which are incorporated by reference in their entirety.

### TECHNICAL FIELD

The present disclosure relates to a novel method of making Copper (I) 5-Nitrotetrazolate (commonly referred to as DBX-1), and to the use of bis(ethylenediamine) copper (II) nitrotetrazolate in making Copper (I) 5-Nitrotetrazolate.

### BACKGROUND

Explosive devices typically comprise a secondary explosive material, which provides the main explosive reaction, and a primary explosive material, which is used to initiate the secondary explosive material.

Historically, lead-based primary explosives such as lead-azide have been commonly used. However, it is now desirable to avoid lead-based explosives for environmental reasons, particularly owing to legislative requirements, which may prohibit used of lead-based primary explosives and precursor materials altogether in the future.

One promising lead-free primary explosive is Copper (I) 5-Nitrotetrazolate, commonly referred to as DBX-1.

Current options for production of DBX-1 involve isolation of an intermediate material sodium 5-nitrotetrazolate (NaNT), which is reduced with a cupric salt to produce DBX-1. The methods produce good yields, but the intermediate material is a sensitive, explosive material, and the method is cumbersome. Some methods require the presence of DBX-1 seed material for the process to be successful.

It is against this background that the forgoing method has been developed.

### SUMMARY OF THE INVENTION

The invention related to a method of making Copper (I) 5-Nitrotetrazolate comprising a) reacting bis(ethylenediamine) copper (II) nitrotetrazolate with acid to form a reaction product; and b) reducing the reaction product of a) to form copper (I) 5-Nitrotetrazolate. By virtue of this method, and in particular the use of bis(ethylenediamine) copper (II) nitrotetrazolate, Copper (I) 5-Nitrotetrazolate can be synthesised quickly and simply, without the need to work with a sensitive intermediate.

The reaction product of a) may comprise an intermediate, and step b) may be carried out without isolating the intermediate from the reaction product. This avoids the need for an additional isolation step, making the process fast and simple, and avoids the need to handle an isolated intermediate, which may be volatile and/or hazardous. This improves safety of the process. The intermediate may comprise decomposed bis(ethylenediamine) copper (II) nitrotetrazolate.

Step b) may comprise reacting the reaction product of step a) with a reducing agent. The reducing agent may for example be sodium ascorbate or ascorbic acid.

The acid used in step a) may be a strong acid, optionally nitric acid or sulfuric acid and preferably nitric acid.

Step a) may comprise heating the mixture to a first reaction temperature. Step b) may comprise heating the mixture to a second reaction temperature. The first and/or second reaction temperatures may be above room temperature. The first reaction temperature may be different to the second reaction temperature. This allows each of steps a) and b) to be carried out at respective optimum temperatures. Preferably, the first reaction temperature is lower than the second reaction temperature.

Step b) may comprise reducing the reaction product of step a) without addition of a cupric salt. Surprisingly, the inventors found that reduction of the intermediate could occur without the need for a cupric salt, and avoiding addition of a cupric salt in step b) improves purity of the final DBX-1 product.

Alternatively, step b) may comprise reacting the reaction product of step a) with a cupric salt. In this case, the cupric salt may be copper chloride or copper sulphate, preferably copper chloride. The cupric salt may be provided in an approximately 1:1 molar ratio with the bis(ethylenediamine) copper (II) nitrotetrazolate.

The invention also extends to the use of bis(ethylenediamine) copper (II) nitrotetrazolate in making Copper (I) 5-Nitrotetrazolate, and to a method of making Copper (I) 5-Nitrotetrazolate, the method comprising utilizing bis(ethylenediamine) copper (II) nitrotetrazolate.

The invention extends further to Copper (I) 5-Nitrotetrazolate made according to the method described above, and to a primary explosive comprising said Copper (I) 5-Nitrotetrazolate.

### BRIEF DESCRIPTION OF THE DRAWINGS

The present invention will now be described by way of non-limiting examples with reference to the following figures, in which:

FIG. 1 is a graph showing the results of a fourier transform infrared spectroscopy (IR) analysis carried out on a DBX-1 material prepared using the method described herein according to Example 1; and

FIG. 2 is a graph showing the results of a differential scanning calorimetry (DSC) analysis carried out on a DBX-1 material prepared using the method described herein according to Example 1.

### DETAILED DESCRIPTION

According to the present method, DBX-1 is made using bis(ethylenediamine) copper (II) nitrotetrazolate, as will now be described.

The method comprises two stages. In a first stage, Stage a), bis(ethylenediamine) copper (II) nitrotetrazolate is reacted with an acid. This causes the bis(ethylenediamine) copper (II) nitrotetrazolate to decompose. The resulting reaction product is a ‘decomposed product’ and includes an intermediate comprising decomposed bis(ethylenediamine) copper (II) nitrotetrazolate. In a second stage, Stage b), the reaction product of Stage a) is mixed with a reduction agent, and is reduced to produce DBX-1.

Between Stage a) and Stage b), there is no need to isolate the intermediate: instead, the entire reaction product of Stage a) can be used directly in Stage b). Where the process is a batch process, this means the same vessel can be used for the reaction mixtures of Stages a) and b), and the cupric salt and reduction agent can, if desired be added directly to the vessel containing the reaction product of Stage a).

In Stage a), the bis(ethylenediamine) copper (II) nitrotetrazolate and acid may be combined with a solvent.

Considering the various reactants in more detail:

The bis(ethylenediamine) copper (II) nitrotetrazolate may be made using any suitable method, such as the method described in GB1519796A, for example the method described in Example 1 of GB1519796A, which is hereby incorporated by reference. In this case, the bis(ethylenediamine) copper (II) nitrotetrazolate may be purified prior to use by recrystallisation using water. Bis(ethylenediamine) copper (II) nitrotetrazolate is relatively safe to handle (in particular safer and easier to purify than sodium 5-nitrotetrazolate). The bis(ethylenediamine) copper (II) nitrotetrazolate may be provided as a solution, preferably an aqueous solution, for example having a concentration between approximately 1 mmol and approximately 25 mmol, preferably between approximately 2 mmol and approximately 10 mmol, for example approximately 5 mmol.

The acid may be any suitable acid capable of decomposing the bis(ethylenediamine) copper (II) nitrotetrazolate, such as nitric acid, sulfuric acid, hydrochloric acid, perchloric acid, acetic acid. Nitric acid may be particularly preferred. The acid may be provided in solution, such as an aqueous solution, for example having a concentration between approximately 1 M and approximately 25 M, preferably between approximately 2 M and approximately 10 M, for example approximately 5 M.

The solvent may be any suitable solvent, such as water, dimethyl sulfoxide (DMSO) or another polar organic solvent.

The reducing agent may be any agent capable of reducing the intermediate product, such as ascorbic acid or sodium ascorbate. The reducing agent may be provided in solution, such as an aqueous solution, for example having a concentration between approximately 0.1 M and approximately 10 M, preferably between approximately 0.5 M and approximately 5 M, for example approximately 1 M.

The reagents may be provided in approximately stoichiometric proportions. The acid is provided in at least a sufficient quantity to breakdown all the bis(ethylenediamine) copper (II) nitrotetrazolate in Stage a), and the reducing agent is provided in at least a sufficient quantity to reduce the intermediate in Step b).

Relative proportions of the various reagents may be as follows:

| | | |
|---|---|---|
| Reagent | Amount | Concentration |

| | | | |
|---|---|---|---|
| Bis(ethylenediamine) copper (II) | 2.05 | g | 5 mmol |
| nitrotetrazolate | | | |
| Acid - nitric acid | 4.2 | ml | 5M |

| | | |
|---|---|---|
| Solvent - water | 10 ml/10 g | n/a |

| | | | |
|---|---|---|---|
| Reducing agent - sodium ascorbate | 4 | g | 1M |

Considering the process steps in more detail:

In Stage a), a vessel containing a mixture of the solvent and the bis(ethylenediamine) copper (II) nitrotetrazolate is heated to a first reaction temperature. The first reaction temperature may be any temperature suitable to facilitate the breakdown of the bis(ethylenediamine) copper (II) nitrotetrazolate, for example between about 40° C. and about 120° C., preferably between about 50° C. and about 90° C., most preferably about 70° C., subject to solvent choice. The acid is added (optionally dropwise) to the mixture.

The reactants are then allowed to react to form the reaction product, including the intermediate.

At this stage, there is no need to isolate the intermediate, and Stage b) can proceed directly from Stage a).

In Stage b), the product of Stage a) is heated to a suitable second reaction temperature. The second reaction temperature may be any temperature suitable to facilitate the reduction reaction, for example between about 60° C. and about 150° C., preferably about 80° C. to 100° C. and most preferably about 90° C.

Next, the reducing agent is added while maintaining the mixture at the second reaction temperature. The reducing agent may be added in two stages. For example a first, relatively small amount of reducing agent may first be added, then the mixture may be stirred, following which a second, relatively large amount of reducing agent may be added, and the mixture stirred again.

In one particular example, the reducing agent is added as follows:
a first amount comprising 25% of the total amount of reducing agent is added dropwise over 10 minutes;
the mixture is stirred for 50 minutes;
a second amount comprising 75% of the total amount of reducing agent is added dropwise over 10 minutes;
the mixture is stirred for 10 minutes.

The mixture is then allowed to cool to room temperature.

The resulting precipitate is filtered (for example under vacuum), washed (for example with water and IPA), and the final DBX-1 product is isolated.

The resulting DBX-1 is an effective primary explosive. The process is simpler and safer than known processes for the production of DBX-1, because there is no need to isolate, purify and handle a sensitive intermediate such as sodium 5-nitrotetrazolate.

According to the present method, it is not necessary to add a cupric salt to the mixture during the reduction of stage b). Such a cupric salt may optionally be included if desired, but a particular advantage of avoiding the cupric salt is that the purity of the final product is improved, compared to process in which a cupric salt is required for the reduction stage.

Where a cupric salt is added, the cupric salt may be any suitable cupric salt, such as copper chloride, copper bromide or copper sulphate. The cupric salt may be provided in solution, such as an aqueous solution, for example having a concentration between approximately 1 mmol and approximately 25 mmol, preferably between approximately 2 mmol and approximately 10 mmol, for example approximately 5 mmol. A molar ratio of the cupric salt to the bis(ethylenediamine) copper (II) nitrotetrazolate is preferably approximately 1:1, for example between approximately 1:0.9 and approximately 1:1.1.

### Example 1

### Making Bis(Ethylenediamine) Copper (II) Nitrotetrazolate

Bis(ethylenediamine) copper (II) nitrotetrazolate was made according to the process described in Example 1 of GB1519796A. The resulting material was purified by recrystallisation using water. A 5 mmol aqueous solution of the bis(ethylenediamine) copper (II) nitrotetrazolate was then produced.

### Stage a)

The following components were mixed to produce a reaction product comprising an intermediate of decomposed Bis(ethylenediamine) copper (II) nitrotetrazolate:
2.05 g of the 5 mmol aqueous solution of Bis(ethylenediamine) copper (II) nitrotetrazolate was added to 100 ml of water at 70° C.;
4.2 ml of 5M HNO₃ was added dropwise to the above solution.

### Stage b)

The entire reaction product of stage a) was mixed with the following components, with no isolation of the intermediate from the reaction product:
The reaction product of stage a) was heated to 90° C.;
1 g of 1M sodium ascorbate was added dropwise over 10 minutes;
The solution was stirred for 50 minutes;
A further 3 g of 1M sodium ascorbate was added dropwise over 10 minutes;
The mixture was stirred for 10 minutes, then allowed to cool to room temperature.

### Isolation of DBX-1

The DBX-1 material was isolated as follows:
The precipitate from stage b) was filtered under vacuum;
The precipitate was washed with cold water then cold IPA;
0.7345 g of DBX-1 was isolated as a red crystalline powder.

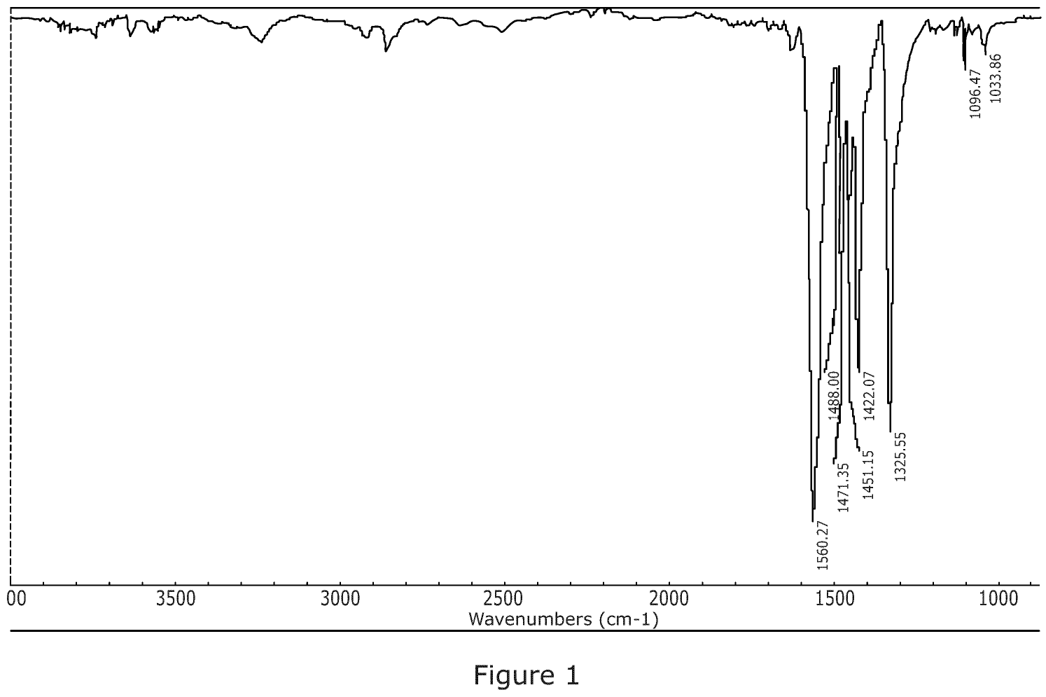

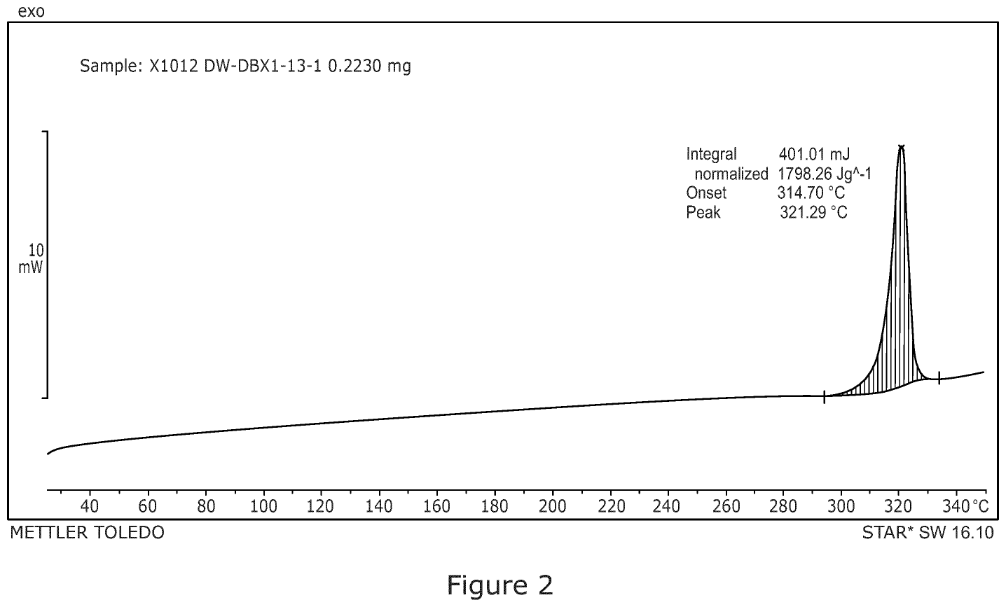

FIGS. 1 and 2 show the results of IR analysis and (DSC) analysis carried out on the final product produced by Example 1, and show that the product is DBX-1.

The yield of the process of Example 1 was found to be 83%.

Prior art methods that require the isolation of an intermediate product before addition of a reducing agent and cupric salt, offer higher yield. Such an isolation step has been found to be unnecessary in the present method. Although this may reduce the overall yield, it has other advantages: in particular, the process described is faster and simpler and also safer, since it does not require isolation and subsequent handling of a sensitive intermediate.

### Example 2

Example 2 was substantially the same as Example 1, except that a cupric salt was included in the mixture for Stage b).

### Making Bis(Ethylenediamine) Copper (II) Nitrotetrazolate

Bis(ethylenediamine) copper (II) nitrotetrazolate was made according to the process described in Example 1 of GB1519796A. The resulting material was purified by recrystallisation using water. A 5 mmol aqueous solution of the bis(ethylenediamine) copper (II) nitrotetrazolate was then produced.

### Stage a)

The following components were mixed to produce a reaction product comprising an intermediate of decomposed Bis(ethylenediamine) copper (II) nitrotetrazolate:
2.05 g of the 5 mmol aqueous solution of Bis(ethylenediamine) copper (II) nitrotetrazolate was added to 20 ml of water at 70° C.;
4.2 ml of 5M HNO₃ was added dropwise to the above solution.

### Stage b)

The entire reaction product of stage a) was mixed with the following components, with no isolation of any intermediate from the reaction product:
0.67 g of 5 mmol copper (II) chloride aqueous solution was added to reaction product of stage a);
The mixture was heated to 90° C.;
1 g of 1M sodium ascorbate was added dropwise over 10 minutes;
The solution was stirred for 50 minutes;
A further 3 g of 1M sodium ascorbate was added dropwise over 10 minutes;
The mixture was stirred for 10 minutes, then allowed to cool to room temperature.

### Isolation of DBX-1

The DBX-1 material was isolated as follows:
The precipitate from stage b) was filtered under vacuum;
The precipitate was washed with cold water then cold IPA;
1.15 g of DBX-1 was isolated as a red crystalline powder.

The yield of the process of Example 2, which includes the presence of a cupric salt in stage b) was found to be 65%, i.e. lower than the yield of Example 1, which did not include the presence of a cupric salt in stage b).

Variations of the invention described above will be apparent to the skilled person without deviating from the scope of the appended claims.

1. A method of making Copper (I) 5-Nitrotetrazolate comprising:
a) reacting bis(ethylenediamine) copper (II) nitrotetrazolate with acid to form a reaction product; and
b) reducing the reaction product of a) to form copper (I) 5-Nitrotetrazolate.

2. The method of claim 1, wherein the reaction product of a) comprises an intermediate, and step b) is carried out without isolating the intermediate from the reaction product.

3. The method of claim 1, wherein step b) comprises reacting the reaction product of step a) with a reducing agent.

4. The method of claim 3, wherein the reducing agent is sodium ascorbate or ascorbic acid.

5. The method of claim 1, wherein the acid is nitric acid or sulfuric acid.

6. The method of claim 1, wherein step a) comprises heating the mixture to a first reaction temperature, and step b) comprises heating the mixture to a second reaction temperature, optionally wherein the first reaction temperature is different to the second reaction temperature, preferably wherein the first reaction temperature is lower than the second reaction temperature.

7. The method of claim 1, wherein step b) comprises reducing the reaction product of step a) without addition of a cupric salt.

8. The method of claim 1, wherein step b) comprises reacting the reaction product of step a) with a cupric salt.

9. The method of claim 8, wherein the cupric salt is copper chloride or copper sulphate.

10. The method of claim 1, wherein step b) takes place subsequent to step a).

11. (canceled)

12. (canceled)

13. Copper (I) 5-Nitrotetrazolate made according to the method of claim 1.

14. A primary explosive comprising the Copper (I) 5-Nitrotetrazolate of claim 13.

15. The method of claim 5, wherein the acid is nitric acid.

16. The method of claim 9, wherein the cupric salt is copper chloride
